(19) United States
(12) Patent Application Publication
(10) Pub. No.: US 2003/0228261 A1
(43) Pub. Date: Dec. 11, 2003

(54) Light emitting microorganisms and cells for diagnosis and therapy of diseases associated with wounded or inflamed tissue

(76) Inventors: Aladar Szalay, Highland, CA (US); A. Douglas Will, Mammoth Lakes, CA (US)

Correspondence Address: INTELLECTUAL PROPERTY / TECHNOLOGY LAW, PO BOX 14329, RESEARCH TRIANGLE PARK, NC, 27709, US

(21) Appl. No.: 10/163,763

(22) Filed: Jun. 5, 2002

(51) Int. Cl. A61K 49/00; C12Q 1/70; C12Q 1/68; A61K 48/00

(52) U.S. Cl. 424/9.34; 435/5; 435/6; 424/93.2

(57) ABSTRACT

A method for using a microorganism or cell containing a DNA sequence encoding a detectable protein or a protein capable of inducing a detectable signal, e.g., a luminescent or fluorescent protein for the preparation of a diagnostic composition for diagnosis and/or visualization of wounded or inflamed tissue or a disease associated therewith. Further, the microorganism or cell may additionally contain an expressible DNA sequence encoding a protein suitable for therapy, e.g. an enzyme causing cell death or digestion of debris.

### BACKGROUND OF THE INVENTION

1. Field of Invention

The present invention relates to the use of a microorganism or cell containing a DNA sequence encoding a detectable protein or a protein capable of inducing a detectable signal, e.g. a luminescent or fluorescent protein, for the preparation of a diagnostic composition for diagnosis and/or visualization of wounded or inflamed tissue or a disease associated therewith. The present invention also relates to therapeutic uses wherein said microorganism or cell additionally contain an expressible DNA sequence encoding a protein suitable for therapy, e.g. an enzyme causing cell death or digestion of debris.

2. Description of the Prior Art

Bacteremia may arise from traumatic injuries and surgical procedures as well as from physiological functions, such as chewing or tooth brushing. Blood cultures taken before and after invasive procedures and physiological functions from healthy human subjects show that while the premanipulation blood samples are sterile, bacteria are present in the blood in varying frequencies depending on the procedures. A potential consequence of bacteremia is colonization of susceptible sites. However, despite the occurrence of transient bacteremias, only a certain percentage of high-risk patients develop bacterial colonization of potentially susceptible sites. A number of investigators have suggested that bacteria from the blood circulation can colonize inflamed tissues in animal models and on the surface of implanted materials. The inconsistency in the pathological changes in humans following a bacteremia may also be due to the resistance of host immune system, the variability in the concentration of bacteria in the blood subsequent to different bacteremia events, and the virulence of any given bacterial strain.

A number of investigators have focused on the nature of the implanted materials as the factor that influences the ability of bacteria to adhere. Materials such as sutures and surgical clips which are used for closure of wounds, are potential sites of bacterial colonization. Infection of these materials may impede wound healing and/or place patients at increased risk of secondary infections. A variety of wound closure materials have been manufactured with varying affinities for bacteria. Certain wound closure materials, such as braided sutures, have been associated with a higher incidence of infection. The multifilament nature of this type of suture material lends itself to increased susceptibility to bacterial colonization as well as causing a wicking effect that allows penetration of bacteria across the tissues. Mere permanent implantable materials have demonstrated a similar affinity for bacteria. Prosthetic heart valves and joints may be at increased risk of bacterial colonization. It is commonly believed that this higher susceptibility is caused by the inherent ability of bacteria to adhere more readily to the implant surfaces. An alternative explanation may be that inflammation in the tissues surrounding the implants provides an environment that is more suitable for bacterial colonization. In addition to these given possibilities, another factor that may influence the susceptibility of a site, with regards to colonization with bacteria could be the degree of inflammatory status of the affected tissues. Implanted materials may create transient or chronic sites of inflammation in the body.

Presence of implanted materials is not a requirement for bacterial colonization. Alteration of natural anatomical structures that may arise from disease conditions may produce surfaces that are easier to colonize by bacteria. It had been suggested that for the occurrence of infective endocarditis (E), the valve surface must be altered in order to produce a suitable site for bacterial attachment and colonization. Additionally, the microorganisms have to reach this site and adhere, since it is not possible to produce IE in experimental animals with injections of bacteria unless the valvular surface is damaged. Lesions with high turbulence create conditions that lead to bacterial colonization, whereas defects with a large surface area or low flow are seldom implicated in IE.

However, so far, it could not be proven that transient bacteremias actually cause colonization of inflamed or wounded tissue, since there was no model available allowing the tracing of bacteria in a living organism, i.e. allowing to explain the temporal and spatial relationship between bacterial infections and diseased tissue sites. Moreover, unfortunately, so far the early diagnosis and therapy of inflamed or wounded tissues or diseases associated therewith, e.g., an atherosclerotic disease, endocarditis, pericarditis etc., are unsatisfactory.

Therefore, it is the object of the present invention to provide a means for the efficient and reliable diagnosis as well as the therapy of wounded or inflamed tissue or a disease associated therewith which overcomes the disadvantages of the diagnostic and therapeutic approaches presently used.

### SUMMARY OF THE INVENTION

According to the present invention this is achieved by the subject matters defined in the claims. In the experiments leading to the present invention it has been found that inflamed tissues, e.g. near implanted material, permit bacterial colonization. Therefore, it is generally possible to visualize inflamed tissues through use of the system of the present invention described below. It could be shown that expression of genes encoding light-emitting proteins in bacteria provides a genetic tool that allows the tracing of the bacteria in a living host, i.e. the evaluation of the dynamics of an infection process in a living host. The external detection of light-emitting bacteria allowed the inventors to non-invasively study the spatial and temporal relationships between infections and the manifested disease conditions. For generation of the light-emitting bacteria, the bacterial luxab operon was used which encodes the enzyme luciferase which catalyzes the oxidation of reduced flavin mononucleotide (FMNE2), in the presence of the substrate, decanal. This reaction then yields FMN, decanoic acid, water and a photon of light. The light photons can then be captured by radiographs, luminometers, or by low light imagers. Recently, the entire bacterial luxcdabe operon, which encodes the substrate as well as the luciferase enzyme, has been used for detection of bacteria in living animals. The advantage of this system is that it does not require exogenously added substrate, which makes it ideal for in vivo studies.

### BRIEF DESCRIPTION OF THE DRAWINGS

FIG. 1: Visualization of Bacteria Intravenously Injected into Nude Mice

Nude mice were injected with 1×10⁷ attenuated Salmonella typhimurium (A) or 1×10⁷ attenuated Vibrio cholera (B). Both strains were transformed with pLITE201 carrying the lux operon. Photon collection was for one minute 20 min after bacterial injections.

FIG. 2: Visualization of S. typhimurium in the Same Animal over a 5-Day Observation Period

Nude mice were injected with 1×10⁷ attenuated S. typhimurium. On the first observation period, bacteria were disseminated throughout the body of the animal (A). Two days later, bacteria were cleared from the animal with the exception of the incision wound and the ear tag region as indicated by the arrows (B). On day 5, the animal had been able to clear the organism from the wounded regions (C).

FIG. 3: Visualization of V. cholera in the Same Animal over an 8-Day Observation Period Nude mice were injected with 1×10⁷ attenuated V. cholera. On the first observation period, bacteria were visualized in the liver region of the animal (A). Five days later, bacteria were cleared from the entire animal with the exception of the incision wound as indicated by the arrows (B). On day 8, the animal had been able to clear the organism from the wound (C).

FIG. 4: Visualization of V. cholera in an Immunocompetent C57 Mouse

1×10⁷ attenuated V cholera were intravenously injected into the animal. Light-emitting bacteria colonized the ear tag on the forth day after bacterial injection (indicated by the white arrow).

FIG. 5: Visualization of Light Emitting Bacteria in the Liver of Rats

Sprague Dawley rats were intravenously injected with 1×10⁸ attenuated E. coli transformed with the plasmid DNA pLITE201 carrying the luxcdabe operon. Photons were collected immediately after infection for one minute under the low light imager (Night Owl). Light emitting bacteria were visualized in the liver of the whole live animal.

FIG. 6: Colonization of Rat Hearts with Light Emitting Bacteria

Intravenous injection of the rats with 1×10⁸ attenuated E. coli transformed with the plasmid pLITE201 carrying the luxedabe operon did not lead to colonization of the hearts of control animals, which had not been catheterized (A). Similar induction of bacteremias in rats catheterized through the right carotid artery lead to the colonization of the heart with light emitting bacteria (B).

FIG. 7: Detection of Residual Bacteria in the Organs of Rats

Three days following intravenous injection of the rats with 1×10⁸ attenuated E. coli, the hearts, livers, and spleens were excised and cultured overnight. Light emitting bacteria were visualized under the low light imager (Hamamatsu) in all specimens from the catheterized rats (A-C), while in the control animals, bacteria were detected in the liver (A) and spleen (B) but not the heart (C).

### DETAILED DESCRIPTION OF THE PREFERRED EMBODIMENTS

In the studies leading to the present invention, the colonization of wounded and inflamed tissue by bacteria initially present in the circulating blood could be demonstrated and it could be shown that tissues that are irritated by implanted materials such as sutures, wound closure clips and prosthetic devices are more susceptible to bacterial colonization subsequent to bacteremias. The data obtained from experiments with the attenuated S. typhimurium shows that following an intravenous injection, bacteria disseminate throughout the body of the live animals. Therefore, it is reasonable to suggest that the bacteria reach the wounded or inflamed sites via the circulation. These findings described in detail in the examples, below, open the way for (a) designing multifunctional viral vectors useful for the detection of wounded or inflamed tissue based on signals like light emission or signals that can be visualised by MRI and (b) the development of bacterium- and mammalian cell-based wounded or inflamed tissue targeting systems in combination with therapeutic gene constructs for the treatment of diseases associated with wounded or inflamed tissue such as, e.g., an atherosclerotic disease. These systems have the following advantages: (a) They target the wounded or inflamed tissue specifically without affecting normal tissue; (b) the expression and secretion of the therapeutic gene constructs are, preferably, under the control of an inducible promoter, enabling secretion to be switched on or off; and (c) the location of the delivery system inside the tissue can be verified by direct visualisation before activating gene expression and protein delivery. Finally, there are a number of diagnostic methods that could be enhanced or advantageously replaced by the diagnostic approach of the present invention. For example, conventional angiography and MRA techniques and MRA techniques both image blood flowing through the lumen of a vessel to visualize plaque, rather than imaging the plaque directly. MRA is particularly sensitive to turbulence caused by the plaque and, as a result, is often inaccurate. These shortcomings can be overcome by the diagnostic uses of the present invention.

Accordingly, the present invention relates to the use of a microorganism or cell containing a DNA sequence encoding a detectable protein or a protein capable of inducing a detectable signal for the preparation of a diagnostic composition for diagnosis and/or visualization of wounded or inflamed tissue or a disease associated therewith. In addition, said microorganism is also useful for therapy, since following visualization of wounded or inflamed tissue compounds suitable for therapy can be applied, e.g. by topical administration, such as, e.g., acylated iridoid glycosides from Scrophularia nodosa, cortisol, corticosteroid analogs, colchicine, methotrexate, non-steroidal anti-inflammatory drugs (NSAIDs), leflunomide, etanercept, minocycline, cyclosporine, thalidomide, a cytotoxic agent, 6-mercaptopurine, azathioprine, antibiotics or one or more of the proteins listed below.

The present invention also relates to the use of a microorganism or cell containing a DNA sequence encoding a detectable protein or a protein capable of inducing a detectable signal for the preparation of a pharmaceutical composition for the treatment of wounded or inflamed tissue or a disease associated therewith, wherein said micoroorganism or cell furthermore contains one or more expressible DNA sequences encoding (a) protein(s) suitable for the therapy of wounded or inflamed tissue or diseases associated therewith.

Proteins suitable for the therapy of wounded or inflamed tissue or diseases associated therewith include transforming growth factor (TGF-alpha), platelet-derived growth factor (PDG-F), keratinocyte growth factor (KGF) and insulin-like growth factor-1 (IGF-1), insulin-like growth factor-binding proteins (IGFBPs), IL-4, IL-8, endothelin-1 (ET-1), connective tissue growth factor (CTGF), TNF-alpha, vascular endothelial growth factor (VEGF), cyclooxygenase, cyclooxygenase-2 inhibitor, infliximab (a chimeric anti-TNF-alpha monoclonal antibody), IL-10, lipase, protease, lysozyme, pro-apoptotic factor, peroxisome proliferator-activated receptor (PPAR) agonist etc.

Any microorganism or cell is useful for the diagnostic and therapeutic uses of the present invention, provided that it replicates in the organism, is not pathogenic for the organism e.g. attenuated and, is recognized by the immune system of the organism, etc.

In a preferred embodiment, the microorganism or cell contains a DNA sequence encoding a luminescent and/or fluorescent protein. As used herein, the term,, DNA sequence encoding a luminescent or fluorescent protein,, also comprises a DNA sequence encoding a luminescent and fluorescent protein as fusion protein.

In an alternative preferred embodiment of the use of the present invention, the microorganism or cell contains a DNA sequence encoding a protein capable of inducing a signal detectable by magnetic resonance imaging (MRI), e.g. a metal binding protein. Furthermore, the protein can bind a contrasting agent, chromophore, or a compound required for visualization of tissues.

Suitable devices for analysing the localization or distribution of luminescent and/or fluorescent proteins in a tissue are well known to the person skilled in the art and, furthermore described in the literature cited above as well as the examples, below.

Preferably, for transfecting the cells the DNA sequences encoding a detectable protein or a protein capable of inducing a detectable signal, e.g., a luminescent or fluorescent protein, are present in a vector or an expression vector. A person skilled in the art is familiar with examples thereof. The DNA sequences can also be contained in a recombinant virus containing appropriate expression cassettes. Suitable viruses that may be used include baculovirus, vaccinia, sindbis virus, Sendai virus, adenovirus, an AAV virus or a parvovirus, such as MVM or H-1. The vector may also be a retrovirus, such as MoMULV, MoMuLV, HaMuSV, MuMTV, RSV or GaLV. For expression in mammals, a suitable promoter is e.g. human cytomegalovirus “immediate early promoter” (pCMV). Furthermore, tissue and/or organ specific promoters are useful. Preferably, the DNA sequences encoding a detectable protein or a protein capable of inducing a detectable signal are operatively linked with a promoter allowing high expression. Such promoters, e.g. inducible promoters are well-known to the person skilled in the art.

For generating the above described DNA sequences and for constructing expression vectors or viruses which contain said DNA sequences, it is possible to use general methods known in the art. These methods include e.g. in vitro recombination techniques, synthetic methods and in vivo recombination methods as described in Sambrook et al., Molecular Cloning, A Laboratory Manual, 2^{nd} edition (1989) Cold Spring Harbor Laboratory Press, Cold Spring Harbor, N.Y., for example. Methods of transfecting cells, of phenotypically selecting transfectants and of expressing the DNA sequences by using the above described vectors are known in the art.

The person skilled in the art knows DNA sequences encoding luminescent or fluorescent proteins that can be used for carrying out the present invention. During the past decade, the identification and isolation of structural genes encoding light-emitting proteins from bacterial luciferase from Vibrio harveyi (Belas et al., Science 218 (1982), 791-793) and from Vibrio fischerii (Foran and Brown, Nucleic acids Res. 16 (1988), 177), firefly luciferase (de Wet et al., Mol. Cell. Biol. 7 (1987), 725-737), aequorin from Aequorea victoria (Prasher et al., Biochem. 26 (1987), 1326-1332), Renilla luciferase from Renilla reniformis (Lorenz et al., PNAS USA 88 (1991), 4438-4442) and green fluorescent protein from Aequorea victoria (Prasher et al., Gene 111 (1987), 229-233) have been described that allow the tracing of bacteria or viruses based on light emission. Transformation and expression of these genes in bacteria allows detection of bacterial colonies with the aid of the low light imaging camera or individual bacteria under the fluorescent microscope (Engebrecht et al., Science 227 (1985), 1345-1347; Legocki et al., PNAS 83 (1986), 9080-9084; Chalfie et al., Science 263 (1994), 802-805).

Luciferase genes have been expressed in a variety of organisms. Promoter activation based on light emission, using luxAB fused to the nitrogenase promoter, was demonstrated in Rhizobia residing within the cytoplasm of cells of infected root nodules by low light imaging (Legocki et al., PNAS 83 (1986), 9080-9084; O'Kane et al., J. Plant Mol. Biol. 10 (1988), 387-399). Fusion of the lux A and lux B genes resulted in a fully functional luciferase protein (Escher et al., PNAS 86 (1989), 6528-6532). This fusion gene (Fab2) was introduced into Bacillus subtilis and Bacillus megatherium under the xylose promoter and then fed into insect larvae and was injected into the hemolymph of worms. Imaging of light emission was conducted using a low light video camera. The movement and localization of pathogenic bacteria in transgenic arabidopsis plants, which carry the pathogen-activated PAL promoter-bacterial luciferase fusion gene construct, was demonstrated by localizing Pseudomonas or Ervinia spp. infection under the low light imager as well as in tomato plant and stacks of potatoes (Giacomin and Szalay, Plant Sci. 116 (1996), 59-72).

Thus, in a more preferred embodiment, the luminescent or fluorescent protein present in the above described microorganism or cell is luciferase, RFP or GFP.

All of the luciferases expressed in bacteria require exogenously added substrates such as decanal or coelenterazine for light emission. In contrast, while visualization of GFP fluorescence does not require a substrate, an excitation light source is needed. More recently, the gene cluster encoding the bacterial luciferase and the proteins for providing decanal within the cell, which includes luxCDABE was isolated from Xenorhabdus luminescens (Meighen and Szittner, J. Bacteriol. 174 (1992), 5371-5381) and Photobacterium leiognathi (Lee et al., Eur. J. Biochem. 201 (1991), 161-167) and transferred into bacteria resulting in continuous light emission independent of exogenously added substrate (Fernandez-Pinas and Wolk, Gene 150 (1994), 169-174). Bacteria containing the complete lux operon sequence, when injected intraperitoneally, intramuscularly, or intravenously, allowed the visualization and localization of bacteria in live mice indicating that the luciferase light emission can penetrate the tissues and can be detected externally (Contag et al., Mol. Microbiol. 18 (1995), 593-603).

Thus, in an even more preferred embodiment, the microorganism or cell containing a DNA sequence encoding a luciferase additionally contains a gene encoding a substrate for a luciferase.

Preferably, the microorganism is a bacterium. Particularly preferred is attenuated Salmonella thyphimurium, attenuated Vibrio cholerae, attenuated Listeria monocytogenesor E.coli. Alternatively, viruses such as Vaccinia virus, AAV, a retrovirus etc. are also useful for the diagnostic and therapeutic uses of the present invention. Preferably, the virus is Vaccinia virus.

Preferably, the cell for the uses of the present invention is a mammalian cell such as a stem cell which can be autologous or heterologous concerning the organism.

In a further preferred embodiment, the microorganism or cell useful in the present invention contains a ruc-gfp expression cassette which contains the Renilla luciferase (ruc) and Aequorea gfp cDNA sequences under the control of a strong synthetic early/late (PE/L) promoter of Vaccinia or the luxCDABE cassette.

In a preferred use of the microorganisms and cells described above the protein suitable for the therapy of diseases associated with wounded or inflamed tissue like atherosclerotic diseases is an enzyme causing cell death or an enzyme causing the digestion of debris, e.g. in the interior of an atherosclerotic plaque causing the plaque to collapse under the force of the intraluminal blood pressure. Suitable enzymes include a lipase, protease, lysozyme, proapoptotic factor, PPAR-agonist etc. If the inflammatory component of atherosclerosis should be treated suitable compounds are cortisol, corticosteroid analogs, cyclooxygenase and cyclooxygenase-2 inhibitors, colchicine, methotrexate, NSAIDs, leflunomide, etanercept, minocycline, cyclosporine, thalidomide, infliximab, IL-10, 6-mercaptopurine, azathioprine or a cytotoxic agent. Some of these compounds might be in the form of prodrugs.

Accordingly, the protein expressed by a microorganism of the invention can be an enzyme converting an inactive substance (pro-drug) administered to the organism into an active substance.

Preferably, the gene encoding an enzyme as discussed above is directed by an inducible promoter additionally ensuring that, e.g., the conversion of the pro-drug into the active substance only occurs in the target tissue, e.g., an IPTG-, antibiotic-, heat-, pH-, light-, metal-, aerobic-, host cell-, drug-, cell cycle- or tissue specific-inducible promoter. Moreover, the delivery system of the present invention even allows the application of compounds which could so far not be used for therapy due to their high toxicity when systemically applied or due to the fact that they cannot be administered, e.g., intravenously in sufficiently high dosages to achieve levels inside, e.g., sinuses, abscesses or across the blood brain barrier. Such compounds include thalidomide, cytotoxic drugs, antibiotics etc.

Furthermore, the microorganism or cell of the present invention can contain a BAC (Bacterial Artificial Chromosome)or MAC (Mammalian Artificial Chromosome) encoding several or all proteins of a specific pathway, e.g. woundhealing-pathway, such as TNF-alpha, COX-2, CTGF etc. Additionally, the cell can be a cyber cell or cyber virus encoding these proteins.

For administration, the microorganisms or cells described above are preferably combined with suitable pharmaceutical carriers. Examples of suitable pharmaceutical carriers are well known in the art and include phosphate buffered saline solutions, water, emulsions, such as oil/water emulsions, various types of wetting agents, sterile solutions etc.. Such carriers can be formulated by conventional methods and can be administered to the subject at a suitable dose. Administration of the microorganisms or cells may be effected by different ways, e.g. by intravenous, intraperetoneal, subcutaneous, intramuscular, topical or intradermal administration. The preferred route of administration is intravenous injection. The route of administration, of course, depends on the nature of the tissue and the kind of microorganisms or cells contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind and localisation of the tissue, general health and other drugs being administered concurrently.

A preferred therapeutical use is the preparation of a pharmaceutical composition for the treatment of endocarditis, pericarditis, inflammatory bowel disease (e.g. Crohn's disease or Ulcerative colitis), low back pain (herniated nucleus pulposis), temporal arteritis, polyarteritis nodosa or an arthritic disease.

In the past few years, there has been many reports showing evidence for Chlamydia pneumoniae, Heliobacter pylori, CMV, HSV and other infectious agents inside atherosclerotic plaques. The presence of these infectious agents within atherosclerotic plaque suggests that the interior of the plaque is a protected environment that permits replication, otherwise these infectious agents would be cleared by the immune system. Moreover, there is considerable evidence that an inflammatory process is present within the interior of atherosclerotic plaque. Accordingly, it is reasonable to assume that this disease can be diagnosed and treated by the microorganisms or cells of the present invention that after intravenous injection—will penetrate into the atherosclerotic plaque where they start to replicate. After a suitable period of time, the plaque can be imaged using, e.g., light sensitive cameras or suitable MRI equipment. Further, said microorganisms or cells can additionally produce an enzyme, e.g. an enzyme as described above, resulting in the elimination of plaques. Thus, a further preferred use is the diagnosis and treatment of an atherosclerotic disease.

A further preferred use is the diagnosis and treatment of coronary artery disease, peripheral vascular disease or cerebral artery disease. Therapeutic treatments according to the present invention might replace treatments like balloon angioplasty, stent placement, coronary artery bypass graft, carotid endarterectomy, aorto-femoral bypass graft and other invasive procedures. Moreover, plaque in inaccessible regions, such as the basilar and middle cerebral arteries can be treated using the therapeutic approach of the present invention.

For the therapy of wounds, fractures, surgical incisions and burns the microorganisms of the present invention are preferably combined with proteins like transforming growth factor (TGF-alpha), platelet-derived growth factor (PDG-F), keratinocyte growth factor (KGF) and insulin-like growth factor-1 (IGF-1), insulin-like growth factor-binding proteins (IGFBPs), IL-4, IL-8, endothelin-1 (ET-1), connective tissue growth factor (CTGF), TNF-alpha, vascular endothelial growth factor (VEGF), cyclooxygenase, cyclooxygenase-2 inhibitor, infliximab (a chimeric anti-TNF-alpha monoclonal antibody), IL-10, lipase, protease, lysozyme, pro-apoptotic factor, peroxisome proliferator-activated receptor (PPAR) agonist (or contain expressible DNA-sequences encoding said proteins). For the treatment of infectious diseases, the microorganisms of the present invention are preferably applied in combination with antibiotics. For the treatment of auto-immune and inflammatory diseases, including rheumatoid arthritis, inflammatory bowel disease and multiple sclerosis, the microorganisms of the present invention are preferably applied in combination with cortisol, corticosteroid analogs, cyclooxygenase and cyclooxygenase-2 inhibitors, colchicine, methotrexate, NSAIDs, leflunomide, etanercept, minocycline, cyclosporine, thalidomide, infliximab, IL-10, 6-mercaptopurine, azathioprine or a cytotoxic agent. For the therapy of diseases like atherosclerosis, the microorganisms of the present invention are preferably applied in combination with lipases, lysozymes, pro-apoptopic factors, PPAR-agonists (or the corresponding DNA-sequences) or an agent listed above with respect to the treatment of inflammatory diseases. For the treatment of Alzheimer's disease, the microorganisms of the present invention are preferably applied in combination with one or more agents listed above with respect to auto-immune- or inflammatory diseases.

Finally, the above described microorganisms and cells are useful for (a) monitoring the efficacy of an antibiotic regimen, preferably based on light extinction or (b) comparing the resistance of various sutures and implantable materials to bacterial colonization.

The present invention is explained by the following examples.

### EXAMPLE 1

### Materials and Methods

(A)Bacterial Strains

The strains used were a non-pathogenic laboratory strain Escherichia coli, strain DH5α, attenuated Salmonella typhimurium (SL7207 hisG46, DEL407[aroA544::Tn101] and attenuated Vibrio cholerae (Bengal 2 Serotyp 0139, M010 DattRSI).

(B) Plasmid Constructs

The plasmid DNA pLITE201 containing the luxcdabe gene cassette was obtained from Dr. F. Marines (Voisey and Marines, Biotech. 24 (1998) 56-58).

(C) Recipient Animals

Five- to six-week-old male BALB/c nu/nu mice (25-30 g body weight) and Sprague Dawley rats (300-325 g body weight) were purchased from Harlan (Frederick, Md., U.S.A.). CS7BL/6J mice were obtained from Jackson Laboratories (Bar Harbor, Me., U.S.A.). All animal experiments were carried out in accordance with protocols approved by the Lorna Linda University animal research committee. The animals containing recombinant DNA materials and attenuated pathogens were kept in the Loma Linda University animal care facility at biosafety level two.

(D) Detection of Luminescence

Immediately before imaging, the animals were anesthetized with intraperitoneal injections of sodium pentobarbital (Nembutal® Sodium solution, Abbot Laboratories, North Chicago, Ill.; 60 mg/kg body weight). The animals were placed inside the dark box for photon counting and recording superimposed images (ARGUS 100 Low Light Imaging System, Hamamatsu, Hamamatsu, Japan and Night Owl, Berthold Technologies, GmbH and Co. KG, Bad Wildbad, Germany). Photon collection was for one minute from ventral and dorsal views of the animals. A photographic image was then recorded and the low light image was superimposed over the photographic image to demonstrate the location of luminescent activity.

### EXAMPLE 2

### Colonization of Cutaneous Wounds by Intravenously Injected Light Emitting Bacteria in Live Animals

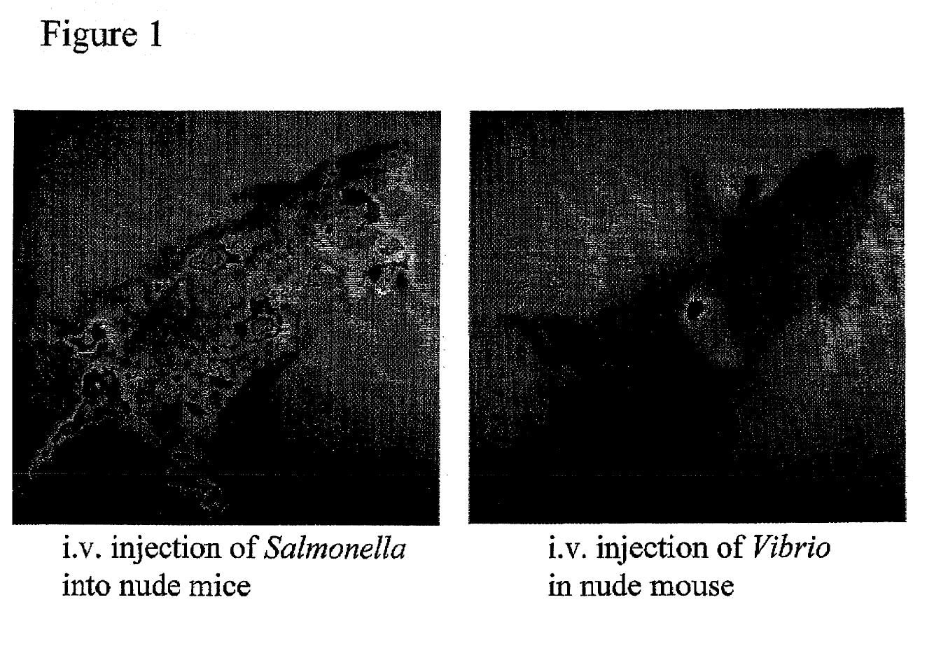

To determine the fate of intravenously injected luminescent bacteria in the animals, 1×10⁷ bacteria carrying the pLITE201 plasmid DNA in 50 μl were injected into the left femoral vein of nude mice under anesthesia. To expose the femoral vein, a 1-cm incision was made with a surgical blade. Following closure of the incision with 6-0 sutures, the mice were monitored under the low light imager and photon emissions were collected for one minute. Imaging of each animal was repeated at various time intervals to study the dissemination of the light-emitting bacteria throughout the body of the animals. It was found that the distribution pattern of light emission following an intravenous injection of bacteria into the mice was bacterial-strain-dependent. Injection of attenuated S. typhimurium caused wide dissemination of the bacteria throughout the body of the animals (FIG. 1A). This pattern of distribution was visible within 5 minutes after bacterial injection and continued to be detected at the one-hour observation period. Injection of attenuated V. cholera into the bloodstream, however, resulted in light emission that was localized to the liver within 5 minutes after bacterial injection and remained visible in the liver at the one-hour observation period (FIG. 1B).

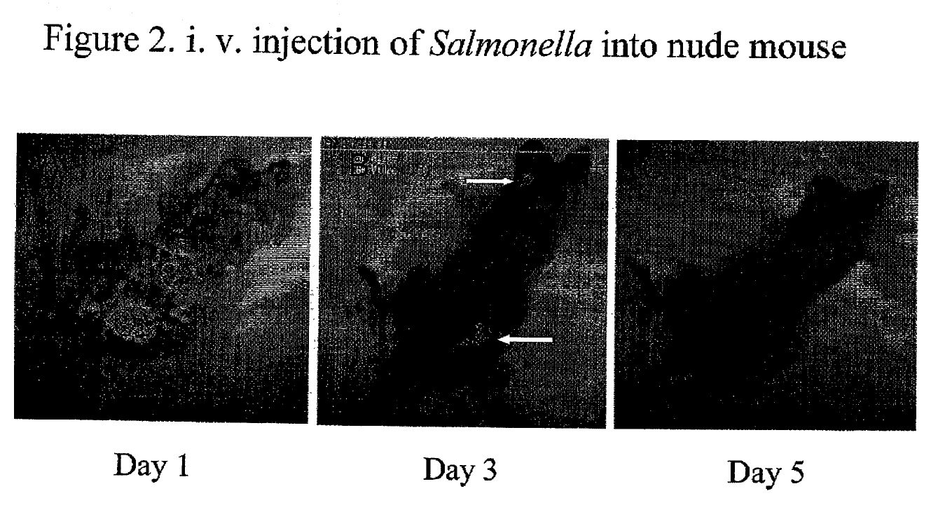

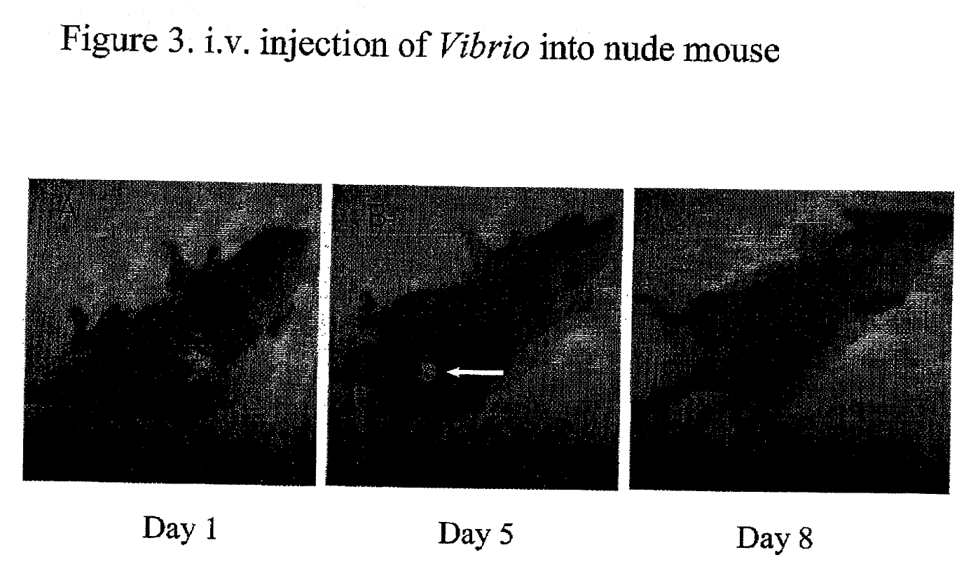

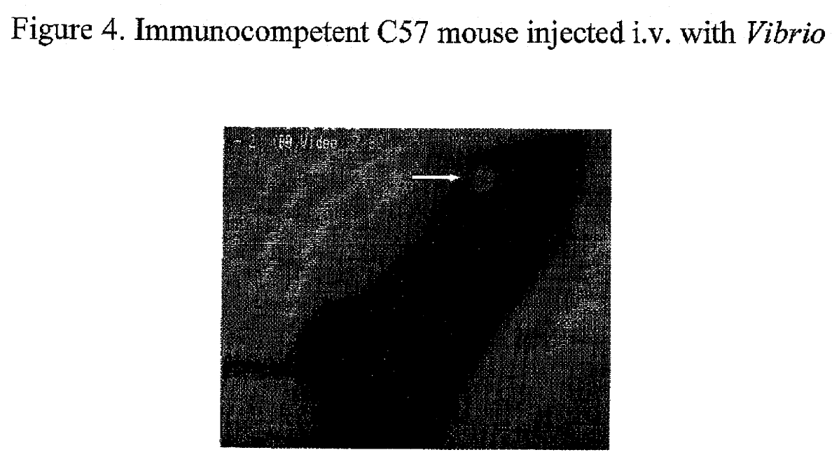

The difference in the bacterial distribution patterns suggests a difference in the interaction of these strains with the host once inside the animal. Imaging the same animals 48 h after bacterial injection showed that all of the detectable light emission from the earlier time had diminished and was eliminated completely from the injected animal with the exception of the inflamed wounded tissues such as the incision wound and the ear tag region. Inflammation in these tissues was identified by their red and edematous appearance. Light emission was detected in the incision wound and/or in the inflamed ear tag region up to 5 to 8 days post injection, which was confirmed by longer photon collection times, i.e. 10 minutes (FIG. 2A-C and FIG. 3A-C). The absence of light emission was not due to the loss of the plasmid DNA or the silencing of gene expression in the bacteria. In other experiments light emission in animals could be consistently detected for up to 50 days. Similar data were obtained in immunocompetent C57BU6J mice (FIG. 4), showing that these observations are not limited to animals with altered immune systems. Careful examination of individually excised organs as well as blood samples from infected animals confirmed the absence of luminescence in these normal uninjured tissues. Furthermore, the experimental data demonstrated that colonization of the injured tissues is a common occurrence in mice. Twenty-four of 29 incision wounds (82.8%) and 12 of 29 ear tags (41.4%) in the mice were colonized by intravenously injected bacteria. Wound colonization by intravenously injected bacteria occurred following injection of V cholera in concentrations as low as 1×10⁵ bacterial cells.

### EXAMPLE 3

### Colonization of Catheterized Rat Hearts Subsequent to Femoral Vein Injection of Light-Emitting Bacteria

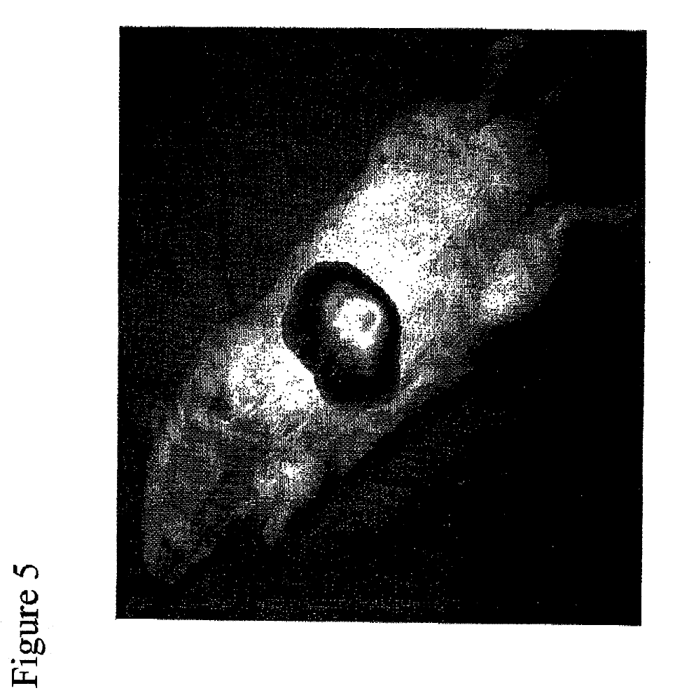

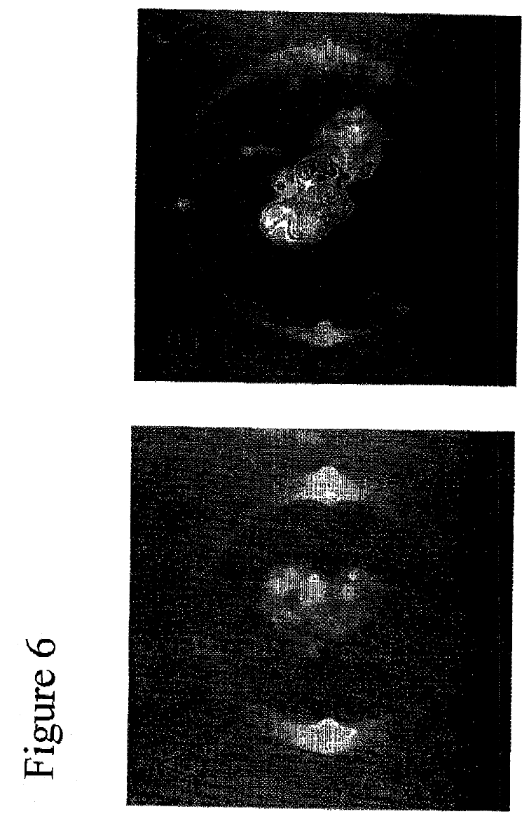

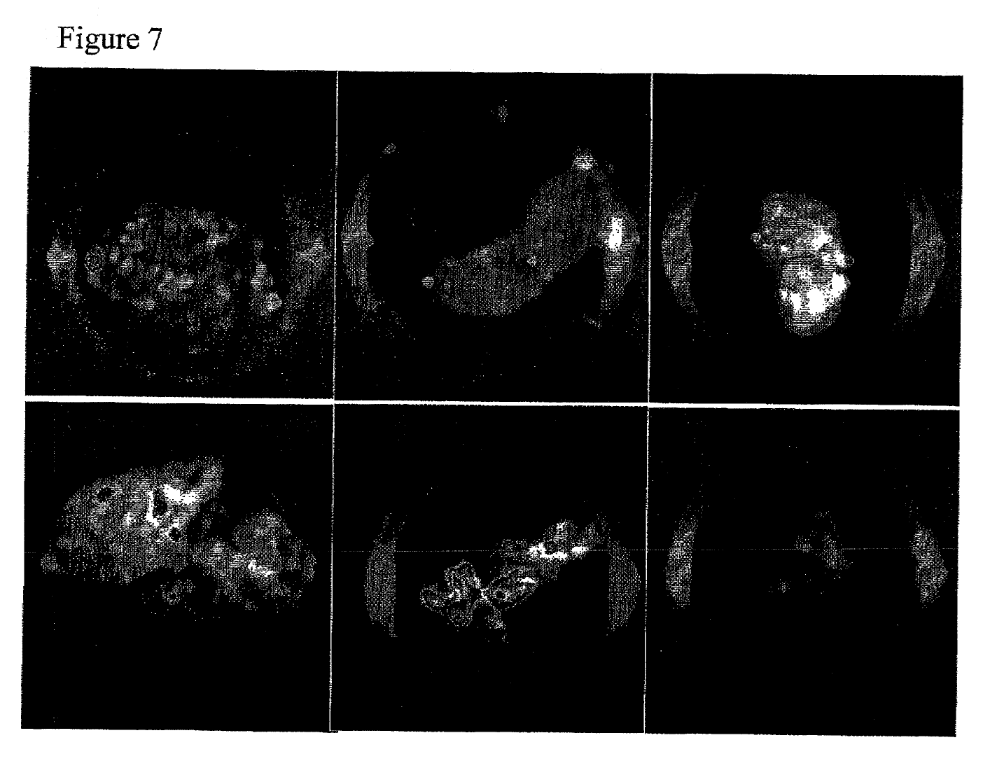

Surgical heart defects were created according to the procedures previously described (Santoro and Levison, Infect. Immun. 19(3) (1978), 915-918; Overholser et al., J. Infect. Dis. 155(1) (1987), 107-112). Briefly, animals were anesthetized with sodium pentobarbital (60 mg/kg i.p.). A midline neck incision was made to expose the tight carotid artery. A polypropylene catheter was introduced and advanced until resistance was met indicating insertion to the level of the aortic valve. The catheter was then secured using a 10-0 suture (AROSurgical Instrument Corporation, Japan) and the incision was closed using 4-0 silk sutures (American Cyanamide Company, Wayne, N.J.). Placement of the catheter causes irritation and subsequent inflammation of the aortic valve (Santoro and Levison, 1978). Control animals did not undergo the catheterization procedure. Bacteremias were induced by injection of 1×10⁸ light-emitting bacterial cells of E. coli via the femoral vein. When observed immediately after infection under the low light imager, bacterial colonization was visible in the liver region (FIG. 5). Three days later, while catheterized animals consistently demonstrated colonization of the heart with light emitting bacteria, control animals showed no sign of light emission from the heart (FIG. 6). To determine if low and undetectable levels of bacteria were present in the tissues, the heart, liver and spleen were excised from each animal and cultured overnight. The livers and spleens of the rats, which are organs that are directly involved in bacterial clearance, in both groups showed presence of light emitting bacteria. Strong light emission was detected in the catheterized heart in contrast to the control heart, which had complete absence of emitted light (FIG. 7). No bacteria were detected on the cultured catheters.

These findings indicate that while light-emitting bacteria injected into the bloodstream via the femoral vein were cleared from normal tissues, injured or inflamed tissues in immunocompromised and immunocompetent animals provided sites that continued to retain bacteria for an extended period of time.

The disclosures of all cited patents and publications are incorporated herein by reference in their entirety for all purposes.

That which is claimed is:

1. A diagnostic composition for diagnosis and/or visualization of wounded or inflamed tissue or a disease associated therewith, comprising:
a microorganism or cell containing a DNA sequence encoding a detectable protein or a protein capable of inducing a detectable signal.

2. A pharmaceutical composition for the treatment of wounded or inflamed tissue or a disease associated therewith, comprising:
a microorganism or cell containing a DNA sequence encoding a detectable protein or a protein capable of inducing a detectable signal and at least one expressible DNA sequences encoding (a) protein(s) suitable for the therapy of wounded or inflamed tissue or a disease associated therewith.

3. The diagnostic composition according to claim 1, wherein the protein capable of inducing a detectable signal is a member selected from the group consisting of a luminescent and a fluorescent protein.

4. The pharmaceutical composition according to claim 2, wherein the protein capable of inducing a detectable signal is a member selected from the group consisting of a luminescent and a fluorescent protein.

5. The diagnostic composition according to claim 1, wherein the protein capable of inducing a detectable signal is a member selected from the group consisting of luciferase, RFP and GFP.

6. The pharmaceutical composition according to claim 2, wherein the protein capable of inducing a detectable signal is a member selected from the group consisting of luciferase, RFP and GFP.

7. The diagnostic composition according to claim 5, wherein the microorganism or cell additionally contains a gene encoding a substrate for a luciferase.

8. The pharmaceutical composition according to claim 6, wherein the microorganism or cell additionally contains a gene encoding a substrate for a luciferase.

9. The diagnostic composition according to claim 1, wherein the protein capable of inducing a detectable signal is a protein selected from the group consisting of: a protein that can induce a signal detectable by magnetic resonance imaging (MRI), a protein having the ability to bind a contrasting agent for visualization of tissue, a protein having the ability to bind a chromophore for visualization of tissue and a protein having the ability to bind to a ligand required for visualization of tissues.

10. The pharmaceutical composition according to claim 2, wherein the protein capable of inducing a detectable signal is a protein selected from the group consisting of: a protein that can induce a signal detectable by magnetic resonance imaging (MRI), a protein having the ability to bind a contrasting agent for visualization of tissue, a protein having the ability to bind a chromophore for visualization of tissue and a protein having the ability to bind to a ligand required for visualization of tissues.

11. The diagnostic composition according to claim 1, wherein the microorganism is a member selected from the group consisting of: a bacterium and a virus.

12. The pharmaceutical composition according to claim 2, wherein the microorganism is a member selected from the group consisting of: a bacterium and a virus.

13. The diagnostic composition according to claim 11, wherein the virus is Vaccinia virus.

14. The diagnostic composition according to claim 11, wherein the bacterium is a member selected from the group consisting of: an attenuated Salmonella thyphimurium, an attenuated Vibrio cholerae, an attenuated Listeria monocytogenes and E. coli.

15. The pharmaceutical composition according to claim 12, wherein the bacterium is a member selected from the group consisting of: an attenuated Salmonella thyphimurium, an attenuated Vibrio cholerae, an attenuated Listeria monocytogenes and E. coli.

16. The diagnostic composition according to claim 1, wherein the cell is a mammalian cell.

17. The pharmaceutical composition according to claim 2, wherein the cell is a mammalian cell.

18. The diagnostic composition according to claim 16, wherein the mammalian cell is selected from the group consisting of: an autologous and heterologous stem cell.

19. The pharmaceutical composition according to claim 17, wherein the mammalian cell is selected from the group consisting of: an autologous and heterologous stem cell.

20. The pharmaceutical composition according to claim 2, wherein the protein suitable for the therapy of wounded or inflamed tissue or a disease associated therewith is selected from the group consisting of: an enzyme causing cell death and an enzyme causing the digestion of debris.

21. The diagnostic composition according to claim 1, wherein the disease is a member selected from the group consisting of: endocarditis, pericarditis, inflammatory bowel disease, low back pain (herniated nucleus pulposis), temporal arteritis, polyarteritis nodosa and an arthritic disease.

22. The pharmaceutical composition according to claim 2, wherein the disease is a member selected from the group consisting of: endocarditis, pericarditis, inflammatory bowel disease, low back pain (herniated nucleus pulposis), temporal arteritis, polyarteritis nodosa and an arthritic disease.

23. The diagnostic composition according to claim 1, wherein the disease is an atherosclerotic disease.

24. The pharmaceutical composition according to claim 2, wherein the disease is an atherosclerotic disease.

25. The diagnostic composition according to claim 1, wherein the disease is selected from the group consisting of; coronary artery disease, peripheral vascular disease and cerebral artery disease.

26. The pharmaceutical composition according to claim 2, wherein the disease is selected from the group consisting of; coronary artery disease, peripheral vascular disease and cerebral artery disease.

27. The diagnostic composition according to claim 1, wherein the diagnosis and/or visualization is carried out by MRI.

28. The pharmaceutical composition according to claim 2, wherein the diagnosis and/or visualization is carried out by MRI.

29. The pharmaceutical composition according to claim 2, wherein the expressible DNA sequences are on a BAC, MAC, cyber cell or cyber virus.

30. The diagnostic composition according to claim 1, wherein the DNA sequence is under the control of an inducible promoter.

31. Use of the diagnostic composition according to claim 1 for monitoring the efficacy of an antibiotic regimen, evaluating the resistance of a suture to bacterial colonization or evaluating the resistance of an implantable material to bacterial colonization.

32. A method for diagnosis and/or visualization of wounded or inflamed tissue or a disease associated therewith, the method comprising:
a) introducing into a microoganism or cell a DNA sequence encoding a detectable protein or a protein capable of inducing a detectable signal;
b) introducing the microoganism or cell into a subject; and
c) monitoring the detectable protein or a protein capable of inducing a detectable signal in the subject.
